(19) United States
(12) Patent Application Publication
(10) Pub. No.: US 2004/0116943 A1
(43) Pub. Date: Jun. 17, 2004

(54) Method and apparatus for endoscopically ligating an elongate tissue structure at multiple sites

(76) Inventors: C. Phillip Brandt, Knoxville, TN (US); G. Clark Greene, Knoxville, TN (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/319,394

(22) Filed: Dec. 13, 2002

(51) Int. Cl. A61B 17/12

(52) U.S. Cl. 606/144

(57) ABSTRACT

A method of ligating a vessel at multiple distant sites through a single surgical opening with a single length of suture filament is disclosed. The method in one embodiment represents an improved endoscopic vessel harvesting technique. An apparatus well suited for performing the method is also disclosed. The apparatus in one embodiment includes a hollow cannula for guiding and advancing loops through the body to the desired ligation sites. Ligation of an elongate tissue structure such as the greater saphenous vein at opposing proximal and distal ends is accomplished by this invention without clips, needles, internal stitches, multiple incisions, or the risks associated with these existing devices and methods.

### FIELD OF THE INVENTION

The present invention relates generally to surgical apparatus and associated methods for ligating and harvesting elongate tissue structures. More particularly, the invention provides a method and apparatus for endoscopically ligating both the proximal and distal ends of a blood vessel using a single suture filament manipulated through a single surgical opening or incision.

### BACKGROUND OF THE INVENTION

Many advanced surgical procedures, such as coronary artery bypass grafting, involve the harvesting, transfer, and grafting of blood vessels to a new location in the body. The greater saphenous vein in the leg, for example, is commonly harvested for use in bypass surgery and other grafting procedures. Early vessel harvesting techniques required an incision spanning the full length of the blood vessel to be harvested. Within this long, narrow surgical field, the surgeon could transect the smaller side branches and remove the main vessel. Unfortunately, the invasive nature of this technique and the large size of the surgical field increased the risk of infection and other complications.

Modern vessel harvesting techniques include the use of an endoscope inserted through a surgical opening or incision to visually inspect and magnify the body cavity around the vessel to be harvested. Related medical instruments may be inserted alongside the endoscope to prepare the vessel for removal. Endoscopic surgery has greatly reduced the risks associated with the earlier, open-field vessel harvesting technique.

Vessel harvesting involves liberating the vessel from surrounding tissue and transecting smaller side branches, tying or ligating the vessel at a proximal site and a distal site, and then transecting the vessel at both sites before it is removed from the body. Endoscopic surgical tools typically work from a centrally located incision or port outward, toward the proximal and distal sites. Once the vessel is prepared, two small counter-incisions can be made at the proximal and distal sites for direct access to the vessel. Although direct ligation using suture filament is generally a secure method, the additional incisions increase the risk of infection, inadvertently puncturing the vessel, and other complications.

Another ligation method involves the use of a long, endoscopic clip applier that can be used to position and apply a clip at the proximal and distal sites. The clip acts as a ligature across the vessel. The clip method requires great skill because it can be difficult to maneuver the applier and clips into the best location to achieve a secure and effective ligation. Multiple clips may be required. The use of clips also introduces the risk that a clip may become loose, may cause injury to the vessel itself, or may cause injury or insult to surrounding structures such as other blood vessels and nearby nerves.

Thus, there is a need in the art for an improved method of ligating a vessel at sites distant from a primary surgical opening without the increased risks associated with the known techniques.

### SUMMARY OF THE INVENTION

The above and other needs are met by the present invention which provides a method and an apparatus for ligating both ends of a blood vessel at proximal and distal sites using a single length of suture filament manipulated through a single surgical opening.

In one aspect of the present invention, the method of ligating an elongate tissue structure at a proximal site and a distal site through a surgical opening positioned between the sites using a suture filament may include the steps of providing a prepared loop at one end of the suture filament secured by a slidable prepared knot, passing an opposing end of the suture filament into the surgical opening and about the tissue structure, tying a slidable field knot about the suture filament with the opposing end to form a field loop about the tissue structure, and advancing the field loop toward the distal site. The method may also include the steps of applying axial tension on the suture filament while stabilizing it when the field loop is surrounding the tissue structure at the distal site so as to close the field loop and tighten the field knot to form a distal ligature, cutting the suture filament near the distal ligature, and transecting the tissue structure between the first ligature and the surgical opening to form a distal end. The next steps may include withdrawing the suture filament through the surgical site and withdrawing the distal end through the surgical site.

The method may further include the steps of inserting the distal end of the tissue structure through the prepared loop, advancing the prepared loop toward the proximal site, and applying axial tension on the suture filament while stabilizing the suture filament and while the prepared loop surrounds the tissue structure at the proximal site so as to close the prepared loop and tighten the prepared knot to form a proximal ligature. The subsequent steps may also include cutting the suture filament proximate the proximal ligature and withdrawing the suture filament through the surgical site.

To complete the harvesting of the tissue, the method may include the additional steps of transecting the tissue structure between the second ligature and the surgical opening to form a proximal end and withdrawing the proximal end through the surgical site.

In another aspect of the method, the step of providing a prepared loop comprises a manufacturing process, which includes stabilizing the suture filament, forming a loop by curling the one end of the suture filament so that it crosses the suture filament, looping the one end of the suture filament about the suture filament in a pattern configured to create the slidable prepared knot, and partially tightening the prepared knot. These steps may be performed by machine or by hand.

In one embodiment of the method, the step of advancing the field loop toward the distal site further includes using an elongate tool to push the field loop. The method of stabilizing the suture filament while the field loop surrounds the tissue structure at the distal site may include stabilizing the elongate tool.

In one embodiment, the elongate tool may be a cannula having first and second ends and a channel therethrough. The method using this cannula may further include threading the opposing end of suture filament through the channel before executing the step of passing the opposing end into the surgical port and about the tissue structure. The step of using an elongate tool may further include using the first end of the cannula. Similarly, the step of stabilizing the suture filament while the field loop surrounds the tissue structure at the distal site may include stabilizing the cannula. In another aspect, the method may include the steps of providing a plurality of cannulae of varying shapes and sizes and selecting the cannula from the plurality of cannulae.

As with the field loop, one embodiment of the method step of advancing the prepared loop toward the proximal site may further include using an elongate tool to push the prepared loop. The step of stabilizing the suture filament while the prepared loop surrounds the tissue structure at the proximal site, likewise, may further include stabilizing the elongate tool. In a method where the elongate tool is a cannula having first and second ends and a channel therethrough, the method may further include threading the opposing end of suture filament through the channel before executing the step of advancing the prepared loop. The step of using an elongate tool may include using the second end of the cannula. And the step of stabilizing the suture filament while the prepared loop surrounds the tissue structure at the proximal site may include stabilizing the cannula in this embodiment. In another aspect, this method may include the steps of providing a plurality of cannulae of varying shapes and sizes and selecting the cannula from the plurality of cannulae.

In another aspect, the method may include providing instructions for tying the slidable field knot. The tying step may include making at least three throws around the suture filament.

In another aspect, the method may include the steps of providing a plurality of suture filaments of varying types and sizes and selecting the suture filament from the plurality of suture filaments.

In an alternative embodiment, the method of the present invention includes tying a first field knot and a second field knot in order accomplish the task of ligating an elongate tissue structure at a proximal site and a distal site through a surgical opening positioned between the sites using a suture filament. In this embodiment, the method may include passing one end of the suture filament into the surgical opening and about the tissue structure, tying a first slidable field knot about the suture filament with the one end to form a first field loop about the tissue structure, advancing the first field loop toward the distal site, and applying axial tension on the suture filament while stabilizing the suture filament and while the first field loop surrounds the tissue structure at the distal site so as to close the first field loop and tighten the first field knot to form a distal ligature. The method may further include cutting the suture filament proximate the distal ligature and withdrawing the suture filament through the surgical site. The second field knot steps may include passing an other end of the suture filament into the surgical opening and about the tissue structure, tying a second slidable field knot about the suture filament with the other end to form a second field loop about the tissue structure, advancing the second field loop toward the proximal site, and applying axial tension on the suture filament while stabilizing the suture filament and while the second field loop surrounds the tissue structure at the proximal site so as to close the second field loop and tighten the second field knot to form a proximal ligature. The steps follow of cutting the suture filament proximate the proximal ligature and withdrawing the suture filament through the surgical site.

The method may further include transecting the tissue structure proximate the first ligature and withdrawing the distal end through the surgical site before the step of tying a second slidable field knot.

Also, the method may further include transecting the tissue structure proximate the second ligature and withdrawing the proximal end through the surgical site.

In one embodiment, the method step of advancing the first field loop toward the distal site further includes using an elongate tool to push the first field loop. The step of stabilizing the suture filament may further include stabilizing the elongate tool. In an embodiment where the elongate tool is a cannula having first and second ends and a channel therethrough, the method further includes threading the one end of suture filament through the channel before executing the step of passing the one end into the surgical port and about the tissue structure. The step of using an elongate tool may include using the first end of the cannula. Similarly, the step of stabilizing the suture filament may further include stabilizing the cannula. In another aspect, the method may include the steps of providing a plurality of cannulae of varying shapes and sizes and selecting the cannula from the plurality of cannulae.

In another embodiment of the method, the step of advancing the prepared loop toward the proximal site includes using an elongate tool to push the prepared loop. The step of stabilizing the suture filament includes stabilizing the elongate tool. Where the elongate tool is a cannula having first and second ends and a channel therethrough, the method includes threading the one end of suture filament through the channel before executing the step of advancing the second field loop. The step of using an elongate tool may include using the second end of the cannula. Likewise, the the step of stabilizing the suture filament may include stabilizing the cannula. In another aspect, the method may include the steps of providing a plurality of cannulae of varying shapes and sizes and selecting the cannula from the plurality of cannulae.

In another aspect, the method may include providing instructions for tying the slidable field knot. The tying step may include making at least three throws around the suture filament.

In another aspect, the method may include the steps of providing a plurality of suture filaments of varying types and sizes and selecting the suture filament from the plurality of suture filaments.

In another aspect of the present invention, an apparatus is provided for ligating an elongate tissue structure at one or more sites through a surgical opening spaced apart from at least one of the one or more sites. The apparatus may include an elongate tool having a first end and a second end, the tool having a channel therethrough, the tool being narrowed toward the ends, and the tool having a length sufficient to extend from the surgical opening to each of the one or more sites. Additionally, the apparatus includes a suture filament having a head end and a tail end. The suture filament may include a prepared loop in the head end secured by a slidable prepared knot tied about the suture filament near the second end of the tool. The suture filament may be threaded through the channel with the tail end extending from the first end of the tool. The tail end may have sufficient length to allow formation of field loop secured by a slidable field knot in the tail end.

In one embodiment, the elongate tool has a central longitudinal axis and the channel lies along the axis. In one embodiment, the elongate tool includes a first conical taper at the first end and a second conical taper at the second end, the conical tapers decreasing in diameter toward the ends. In one embodiment, the elongate tool is a cannula. The sites reached by the apparatus may include a proximal site and a distal site.

In another aspect of the apparatus, the elongate tool may include a temporary anchor positioned and configured to exert a grasp upon the suture filament, thereby preventing movement of the suture filament through the channel. The grasp of the anchor is capable of being overcome by exerting a force upon the suture filament, if the force exceeds a pre-determined threshold. In one embodiment, the temporary anchor is an adhesive spot.

In one embodiment, the sufficient length of the tail end is at least eight inches. In one embodiment, the surgical opening may be a surgical port. In another, it may be a surgical incision.

In an alternative embodiment of the apparatus, no prepared loop in the suture filament is provided. The apparatus for ligating an elongate tissue structure at one or more sites through a surgical opening spaced apart from at least one of the one or more sites in this embodiment includes an elongate tool having a first end and a second end, the tool having a channel therethrough, the tool being narrowed toward the ends, and the tool having a length sufficient to extend from the surgical opening to each of the one or more sites. The apparatus also includes a suture filament threaded through the channel, the suture filament having a head end and a tail end, each of the head and tail ends having sufficient length extending beyond the first and second ends of the tool, respectively, to allow formation of at least one field loop secured by a slidable field knot.

In one embodiment, the elongate tool has a central longitudinal axis and the channel lies along the axis. In one embodiment, the elongate tool includes a first conical taper at the first end and a second conical taper at the second end, the conical tapers decreasing in diameter toward the ends. In one embodiment, the elongate tool is a cannula. The sites reached by the apparatus may include a proximal site and a distal site.

In another aspect of the apparatus, the elongate tool may include a temporary anchor positioned and configured to exert a grasp upon the suture filament, thereby preventing movement of the suture filament through the channel. The grasp of the anchor is capable of being overcome by exerting a force upon the suture filament, if the force exceeds a pre-determined threshold. In one embodiment, the temporary anchor is an adhesive spot.

In one embodiment, the sufficient length of the tail end is at least eight inches. In one embodiment, the surgical opening may be a surgical port. In another, it may be a surgical incision.

These and other objects are accomplished by the method and apparatus disclosed and will become apparent from the following detailed description of a preferred embodiment in conjunction with the accompanying drawings in which like numerals designate like elements.

### BRIEF DESCRIPTION OF THE DRAWING

FIG. 1 is a pictorial view of an apparatus according to one embodiment of the present invention.

FIG. 2 is a side view of an elongate tool with tapered ends according to one embodiment of the present invention.

FIG. 3 is a perspective drawing of a vessel to be harvested and the operative sites according to one embodiment of the present invention.

FIG. 4 is a perspective drawing of a field loop in use, according to one embodiment of the present invention.

FIG. 5 is a perspective drawing of the advancement of an apparatus toward a ligation site, according to one embodiment of the present invention.

FIG. 6 is a perspective drawing of a vessel ligature according to one embodiment of the present invention.

FIG. 7 is a perspective drawing of a prepared loop in use, according to one embodiment of the present invention.

FIG. 8 is a perspective drawing of an elongate tool and a vessel ligature according to one embodiment of the present invention.

### DETAILED DESCRIPTION OF THE INVENTION

Reference is now made to the figures, in which like numerals indicate like elements throughout the several views.

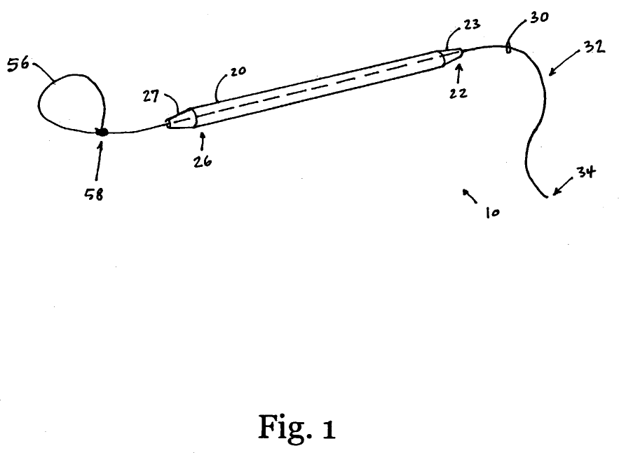

FIG. 1 shows an apparatus 10 according to one embodiment of the present invention. The apparatus 10 includes a suture filament 30 threaded completely through a hollow elongate tool or a cannula 20. A cannula, generally, is a small, elongate tube used for insertion into a body cavity or duct. A cannula may or may not have a consistent shape along its entire length. A cannula may be generally cylindrical, but it may be elliptical or some other shape in cross section. The cannula 20 of the present invention may be described as having two opposing ends, a first end 22 and a second end 26. The diameter of the cannula 20 is narrowed toward the ends 22, 26. The narrowing may take any geometric shape having generally smooth, rounded edges, including shapes such as a hemisphere, a bevel ring, a quarter torus, or a curved shell ring. The narrowing is shaped to facilitate movement of the cannula 20 through body cavities without causing injury to the tissues.

In one embodiment, this narrowing takes the shape of a first conical taper 23 at the first end 22 and a second conical taper 27 at the opposing second end 26. The tapered ends are cone-shaped such that the diameter of the cannula 20 gradually decreases toward the ends 22, 26. The conical tapers 23, 27 are particularly desirable in one embodiment because, as the cannula 20 moves through a body cavity, the leading end (22 or 26) is gradually increasing in diameter. The gradual increase offered by a cone shape tends to move more gently and easily through narrow passages than other, more blunt shapes.

The suture filament 30 in one embodiment includes a prepared loop 56 on one end and a long, un-looped tail 32 at the other end. The prepared loop 56 is made during manufacture in one embodiment, and secured by a pre-tied or prepared knot 58 that is tied around the suture filament 30 during the manufacturing process. Creation of a prepared loop 56 secured by a prepared knot 58 may be done by machine, by hand, or by a combination of the two. The process, generally, includes stabilizing the suture filament 30, forming the prepared loop 56 by curling a free end of the suture filament 30 so that it crosses itself, and looping the free end around the suture filament 30 in an established pattern to create the desired type of prepared knot 58. The prepared knot 58 may be a partially-tightened, slidable knot, tied using any one of a number of established slip knot or slidable knot patterns familiar to those of skill in the art. In one embodiment, the prepared knot 58 may include at least three passes or throws around the suture filament 30 in order to form an acceptably durable knot. A slip knot may be used for suturing in order to facilitate secure and efficient closure of the loop 56 and tightening of the knot 58 when placed in its intended location.

The tail 32 of the suture filament 30 is generally long enough for the user to loop the suture filament 30 around a tissue structure or blood vessel in the surgical field. The tail 32 in one embodiment is twelve to fourteen inches (304 mm to 356 mm) in length, although other lengths are contemplated for use in various environments and procedures. The tail 32 may be measured from the first end 22 of the cannula 20 to the tail end 34 of the suture filament 30. In another embodiment, the tail 32 may be as short as eight inches (203 mm).

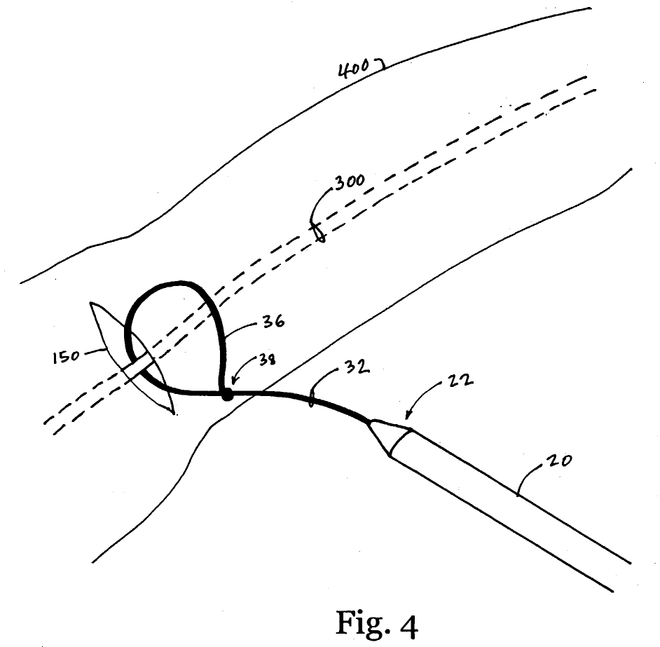

In one embodiment, the user may use the tail 32 to create a field loop 36 by tying a field knot 38 around the suture tail 32, as shown in FIG. 4. The process of creating a field loop 36 secured by a field knot 38 is similar, generally, to the steps described above for creating a prepared loop 56 secured by a prepared knot 58. The field knot 38 may be a partially-tightened, slidable knot, tied using any one of a number of established slip knot or slidable knot patterns familiar to those of skill in the art. In one embodiment, the field knot 38 may include at least three throws around the suture filament 30 in order to form an acceptably durable knot. A slip knot pattern may be used for the field knot 38 in order to facilitate secure and efficient closure of the field loop 36 and tightening of the field knot 38.

In another embodiment, the suture filament 30 does not include a prepared loop on either end. The user may create one or more field loops 36 as needed for a particular procedure.

Any surgical suturing material may be used for the suture filament 30, including natural or synthetic filaments, bio-absorbable or non-absorbable fibers, or any of a variety of available materials suited to the particular need. The suture filament 30 may be a monofilament or it may be composed of multiple strands of filament braided together. In one embodiment, a dyed monofilament, synthetic, absorbable suture material having a U.S.P. suture size designation between 0 (0.071 mm in diameter) and 6-0 (0.015 mm), with size 2-0 (0.029 mm) being a desired size for ligating a greater saphenous vein. Other suture materials and sizes are contemplated for use in various procedures. In one embodiment, a radiopaque marker may be provided on either end of the suture filament 30 to ensure that the knots may be visible on future radiographs.

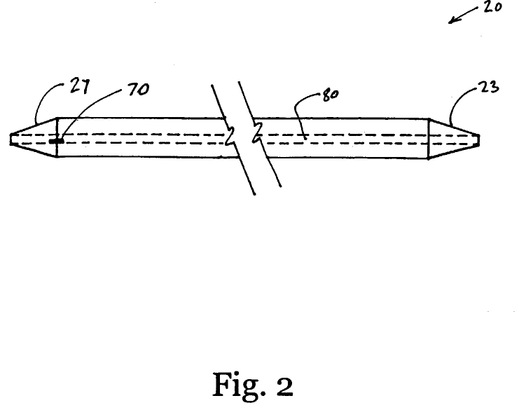

Referring now to FIG. 2, a closer view of an elongate tool or cannula 20 is shown, according to one embodiment of the present invention. The channel 80 through the cannula 20 may coincide with the central longitudinal axis of the cannula 20. The cannula 20 may have a length in one embodiment of between twelve and thirty-six inches (304 mm to 915 mm), with twenty-two inches (559 mm) being a desired size for harvesting a greater saphenous vein in the leg.

The cannula 20 may be constructed from a flexible and durable nylon, plastic, or other suitable biocompatible material. The degree of flexibility desired in a particular cannula 20 will vary depending on the environment and the procedure. The cannula 20 may be disposable or reusable. The cannula 20 may be transparent or opaque, and it may be dyed a particular color or otherwise color-coded. In one embodiment, the cannula 20 may include a measuring scale imprinted on the outer surface. In another embodiment, the cannula 20 may include a series of marks or markers indicating, for example, the preferred insertion depth for certain procedures.

The conical tapers 23, 27 at the opposing ends, in one embodiment, narrow to a size roughly equal to the diameter of the channel 80, as shown. The conical tapers 23, 27 facilitate the safe movement of the cannula 20 into a surgical opening and through body tissue or internal body cavities. Additionally, the conical tapers 23, 27 narrow the cannula ends 22, 26 to facilitate pushing of suture loops into the surgical opening 150 and through internal cavities.

The outer diameter of the cannula 20 may be sized according to the intended use, the surgical field, and other factors related to the procedure being performed. In one embodiment, a desired diameter for the cannula 20 is about 0.16 inches (4 mm).

Inside the cannula 20, the channel 80 may have a diameter that is larger than the diameter of the suture filament 30 to be used, so that the suture filament 30 can slide through the channel 80. The diameter of the channel 80, however, may be sized to provide enough resistance against the suture filament 30 to prevent the suture filament 30 from accidentally or inadvertently sliding through the channel 80.

In another aspect of the apparatus of the present invention, the suture filament 30 may be loosely held in place inside the channel 80 by a temporary anchor 70 or other temporary securing means to keep the suture filament 30 within the cannula 20 until ready for use. The anchor 70 may be a small adhesive spot, as illustrated in FIG. 2, positioned at a location where the anchor 70 will grasp the suture filament 30. The anchor 70 may include an amount of adhesive, for example, in a patch or area of a given size and thickness, such that the anchor 70 will exert enough of a grasp upon the suture filament 30 to keep it from moving, but not so much that the suture filament 30 is permanently anchored. The grasp of the anchor 70 or other securing means may be overcome by exerting a predetermined, threshold level of force upon the suture filament 30. Also, after the threshold force has been applied once and the suture filament 30 has been released, the anchor 70 preferably would no longer exert a grasp on the suture filament 30.

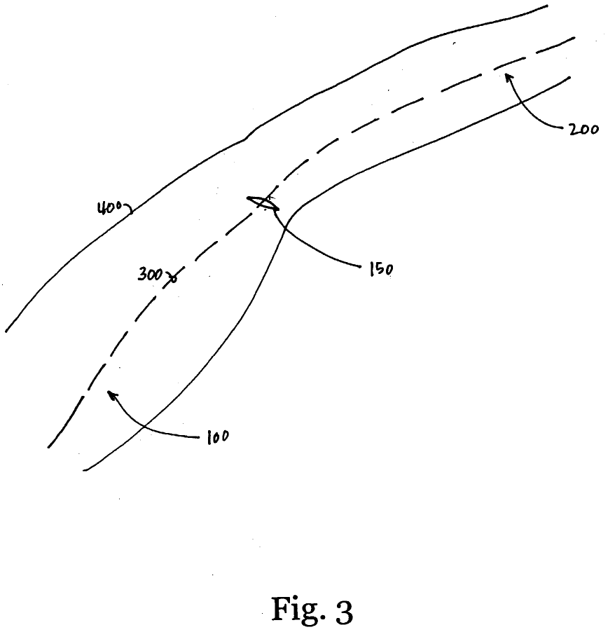

FIG. 3 is an illustration of a surgical field on the body 400 of a patient, showing a vessel 300 in situ to be harvested. The field includes a surgical opening 150 typically formed by making an incision near the vessel 300, near the center of the field. The vessel may be ligated at two or more distant sites, such as the proximal site 100 and distal site 200 shown. The word proximal is used herein to describe an object that is close to the body or to the user, whereas the word distal describes something located away from the body or the user. For example, if the body part in FIG. 3 represents a leg, the surgical opening 150 may be formed near the knee, the proximal site 100 may be close to the hip, and the distal site 200 may be close to the ankle.

The surgical opening 150 may take a variety of forms according to the demands of the particular procedure being performed. The surgical opening 150, for example, may include a surgical port placed within the incision and supported by a hollow tube called a trocar through which the endoscope and related instruments may be placed. In other procedures, the surgical opening 150 may include an incision held open by a spreader or other supportive means. One skilled in the art will appreciate that the method and apparatus of the present invention are well suited for use with any kind of surgical opening 150 capable of facilitating endoscopic surgery.

Method of Ligating a Vessel

In another aspect, the present invention provides a method of ligating any tissue structure. The method works particularly well upon elongate tissue structures such as blood vessels and the like. The method and apparatus may be used to harvest any vessel, including a greater saphenous vein, a radial artery, or a cephalic vein. The method of the present invention allows the user to ligate an elongate tissue structure at multiple sites using a single length of suture filament.

Referring now to FIG. 4, a first step in one embodiment includes passing the end of the tail 32 of the suture filament 30 into the surgical opening 150, under the vessel 300, and back toward the suture filament 30 to form a field loop 36. A field knot 38 tied to the suture filament 30 secures the field loop 36. The field knot 38 may be any one of a number of slip knots or slidable knots familiar to those of skill in the art.

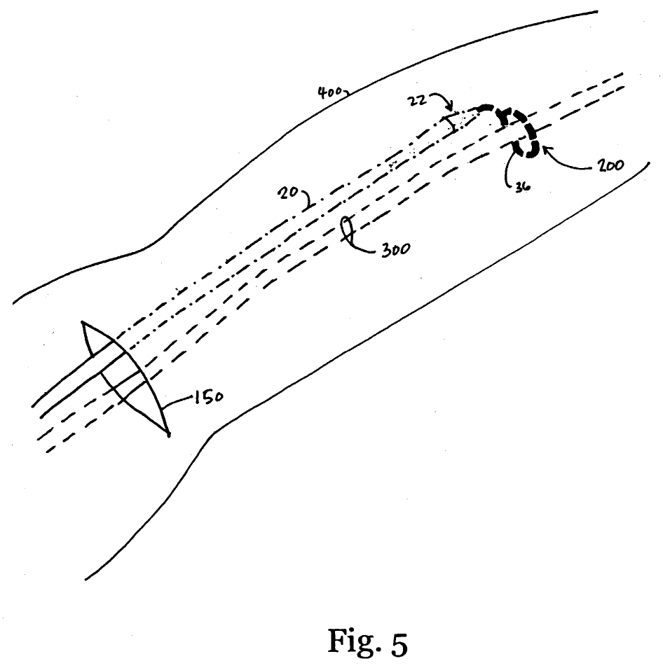

Once the field loop 36 is secure around the vessel 300, a finger or an elongate tool may be used to advance or push the field loop 36 into the surgical opening 150 and toward a desired ligation site. In one embodiment, FIG. 5 shows an elongate tool known as a cannula 20 advancing or pushing the field loop 36 into the surgical opening 150 and along a path near the vessel 300 toward the distal site 200. The field loop 36 slides along the vessel 30 and remains open. In this aspect, in addition to acting as a conduit for the suture filament 30, the tool or cannula 20 serves an additional function as a knot pusher.

In use, the field knot 38 may rest against the elongate tool or the first end 22 of the cannula 20 as the field loop 36 is being advanced or pushed through the surrounding tissue toward the ligation site. There is little or no tension, however, placed on the suture filament 30 during the pushing motion, thus enabling the field loop 36 to remain open 36 and the field knot 38 to remain only partially tightened. In this aspect, the conically tapered shape of the ends 22, 26 of the cannula 20 and the diameter of the channel 80 prevent the premature or unintended tightening of the field knot 38 during pushing.

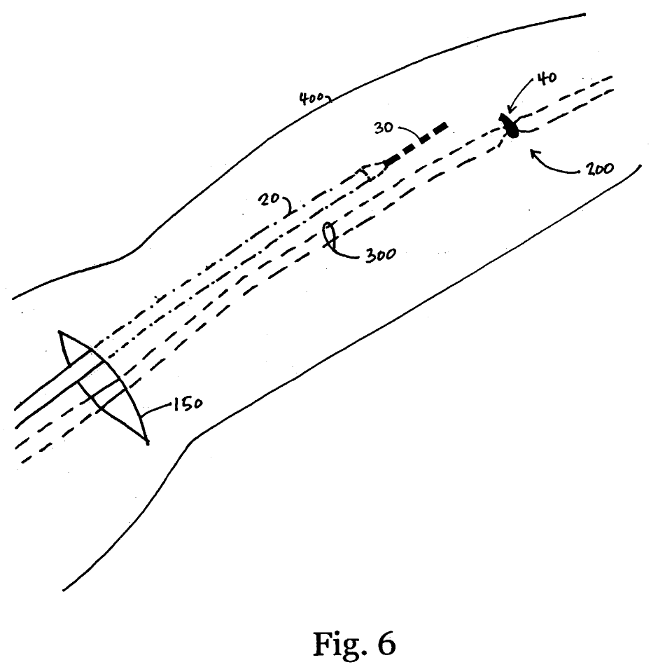

When the field loop 36 has reached the distal site 200 where ligation of the vessel 300 is desired, the user may manually pull or otherwise apply axial tension to the suture filament 30 while stabilizing the cannula 20 in order to close the field loop 36 and tighten the field knot 38, thereby forming a distal ligature 40, as shown in FIG. 6. The suture filament 30 may then be cut, leaving the distal ligature 40 in place and freeing the cannula 20 and suture filament 30 to be withdrawn through the surgical opening 150.

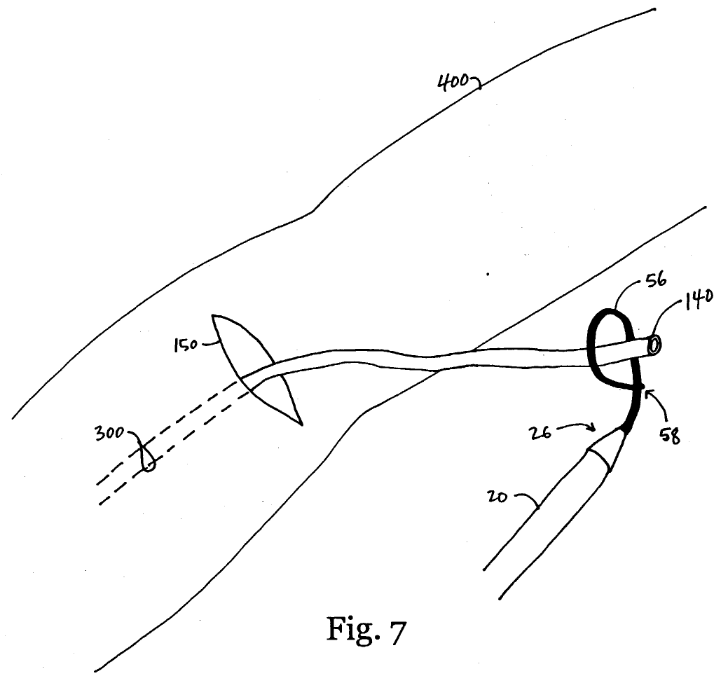

Once the distal ligature 40 is securely in place, the vessel 300 may be transected and withdrawn from the body 400 through the surgical opening 150. As shown in FIG. 7, the prepared loop 56 may be placed over the new distal vessel end 140. In one embodiment, as described above, the suture filament 30 includes a prepared loop 56 at one end, secured by a partially-tightened, slidable, prepared knot 58 tied around the suture filament 30. As shown in FIG. 7, the prepared loop 56 of the suture filament 30 is extending from the second end 26 of the cannula 20. Providing a prepared loop 56 secured by a prepared knot 58 facilitates the prompt capture of the vessel 300 and eliminates the time-consuming step of trying a second field knot during a surgical procedure.

In an alternative embodiment, the user may form a second field loop by passing the suture filament 30 around the vessel 300 and tying a second field knot around the suture filament 30 to secure the loop. In this aspect, the cannula 20 may be used with a length of suture filament 30 for as many loops, knots, and ligatures are needed for a particular procedure.

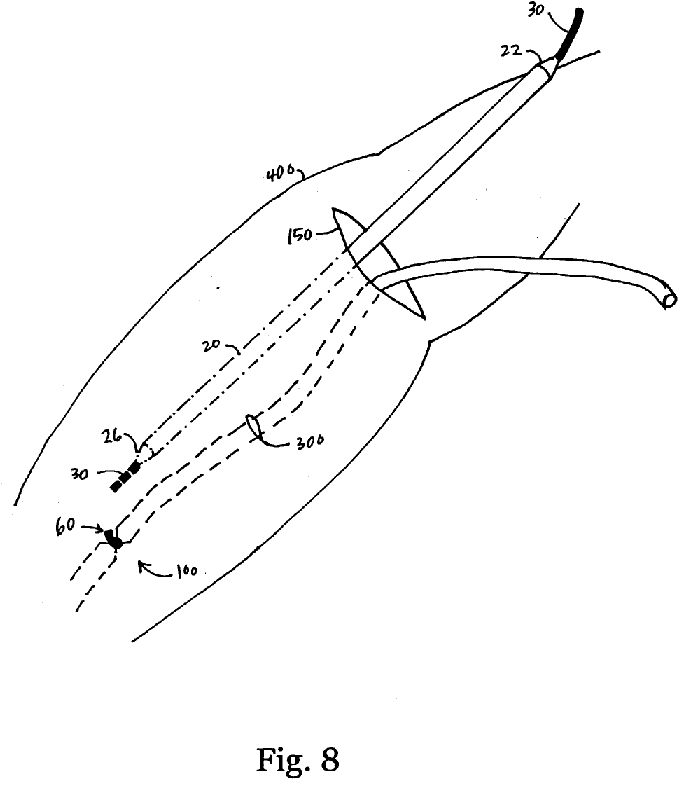

A finger or an elongate tool may also be used to advance or push the prepared loop 56 in a manner similar to that described above and shown in FIG. 5. In one embodiment, an elongate tool known as a cannula 20 may advance or push the prepared loop 56 into the surgical opening 150 and along a path near the vessel 300 toward the proximal site 100. When the prepared loop 56 has reached the proximal site 100 where ligation of the vessel 300 is desired, as shown in FIG. 8, the user may manually pull or otherwise apply axial tension to the suture filament 30 while stabilizing the cannula 20 in order to close the prepared loop 56 and tighten the prepared knot 58, thereby forming a proximal ligature 60.

The suture filament 30 may then be cut, leaving the proximal ligature 60 in place and freeing the cannula 20 and suture filament 30 to be withdrawn through the surgical opening 150. Once the proximal ligature 60 is securely in place, the vessel 300 may be transected and withdrawn from the body 400 through the surgical opening 150.

In another aspect, the method of the present invention may include providing an array of elongate tools or cannulae of different shapes and sizes for the user to choose from, according to the particular procedure being performed. Similarly, in one embodiment, the method may include providing an array of sutures of various types and sizes for the user to choose from, according to the particular procedure being performed. Additionally, the method may include providing instructions for tying a number of field knots, indicating which field knot is particularly well suited to a certain procedure.

Thus, the present invention provides an apparatus and a method of using the apparatus to ligate a vessel 300 at both proximal and distal sites 100, 200 using a single suture filament 30, manipulated through a single surgical opening 150 without clips, needles, or suturing. The invention also provides a method of using a single cannula 20 to manipulate a suture filament 30 to perform multiple ligations.

Although the invention has been described in terms of one or several embodiments, it will be appreciated by those skilled in the art that additions, substitutions, modifications, and deletions not specifically described may be made without departing from the spirit and scope of the invention as defined in the claims.

1. A method of ligating an elongate tissue structure at a proximal site and a distal site through a surgical opening positioned between said sites using a suture filament, said method comprising:
providing a prepared loop at one end of said suture filament secured by a slidable prepared knot tied about said suture filament;
passing an opposing end of said suture filament into said surgical opening and about said tissue structure;
tying a slidable field knot about said suture filament with said opposing end to form a field loop about said tissue structure;
advancing said field loop toward said distal site;
applying axial tension on said suture filament while stabilizing said suture filament and while said field loop surrounds said tissue structure at said distal site so as to close said field loop and tighten said field knot to form a distal ligature;
cutting said suture filament proximate said distal ligature;
transecting said tissue structure between said first ligature and said surgical opening to form a distal end;
withdrawing said suture filament through said surgical site;
withdrawing said distal end through said surgical site;
inserting said distal end of said tissue structure through said prepared loop;
advancing said prepared loop toward said proximal site;
applying axial tension on said suture filament while stabilizing said suture filament and while said prepared loop surrounds said tissue structure at said proximal site so as to close said prepared loop and tighten said prepared knot to form a proximal ligature;
cutting said suture filament proximate said proximal ligature; and
withdrawing said suture filament through said surgical site.

2. The method of claim 1, further comprising:
transecting said tissue structure between said second ligature and said surgical opening to form a proximal end; and
withdrawing said proximal end through said surgical site.

3. The method of claim 1, wherein said step of providing a prepared loop comprises a manufacturing process, said process comprising:
stabilizing said suture filament;
forming a loop by curling said one end of said suture filament so that it crosses said suture filament;
looping said one end of said suture filament about said suture filament in a pattern configured to create said slidable prepared knot; and
partially tightening said prepared knot.

4. The method of claim 3, wherein said manufacturing process steps are performed by machine.

5. The method of claim 3, wherein said manufacturing process steps are performed by hand.

6. The method of claim 1, wherein said step of advancing said field loop toward said distal site further comprises using an elongate tool to push said field loop.

7. The method of claim 6, wherein said step of stabilizing said suture filament while said field loop surrounds said tissue structure at said distal site further comprises stabilizing said elongate tool.

8. The method of claim 6, wherein said elongate tool comprises a cannula having first and second ends and a channel therethrough, said method further comprising:
threading said opposing end of suture filament through said channel before said step of passing said opposing end into said surgical port and about said tissue structure.

9. The method of claim 8, wherein said step of using an elongate tool further comprises using said first end of said cannula.

10. The method of claim 8, wherein said step of stabilizing said suture filament while said field loop surrounds said tissue structure at said distal site further comprises stabilizing said cannula.

11. The method of claim 8, further comprising the steps of:
providing a plurality of cannulae of varying shapes and sizes; and
selecting said cannula from said plurality of cannulae.

12. The method of claim 1, wherein said step of advancing said prepared loop toward said proximal site further comprises using an elongate tool to push said prepared loop.

13. The method of claim 12, wherein said step of stabilizing said suture filament while said prepared loop surrounds said tissue structure at said proximal site further comprises stabilizing said elongate tool.

14. The method of claim 12, wherein said elongate tool comprises a cannula having first and second ends and a channel therethrough, said method further comprising:
threading said opposing end of suture filament through said channel before said step of advancing said prepared loop.

15. The method of claim 14, wherein said step of using an elongate tool further comprises using said second end of said cannula.

16. The method of claim 14, wherein said step of stabilizing said suture filament while said prepared loop surrounds said tissue structure at said proximal site further comprises stabilizing said cannula.

17. The method of claim 14, further comprising the steps of:
providing a plurality of cannulae of varying shapes and sizes; and
selecting said cannula from said plurality of cannulae.

18. The method of claim 1, further comprising:
providing instructions for tying said slidable field knot.

19. The method of claim 1, wherein said step of tying a slidable field knot further comprises at least three throws around said suture filament.

20. The method of claim 1, further comprising the steps of:
providing a plurality of suture filaments of varying types and sizes; and
selecting said suture filament from said plurality of suture filaments.

21. A method of ligating an elongate tissue structure at a proximal site and a distal site through a surgical opening positioned between said sites using a suture filament, said method comprising:
passing one end of said suture filament into said surgical opening and about said tissue structure;
tying a first slidable field knot about said suture filament with said one end to form a first field loop about said tissue structure;
advancing said first field loop toward said distal site;
applying axial tension on said suture filament while stabilizing said suture filament and while said first field loop surrounds said tissue structure at said distal site so as to close said first field loop and tighten said first field knot to form a distal ligature;
cutting said suture filament proximate said distal ligature;
withdrawing said suture filament through said surgical site;
passing an other end of said suture filament into said surgical opening and about said tissue structure;
tying a second slidable field knot about said suture filament with said other end to form a second field loop about said tissue structure;
advancing said second field loop toward said proximal site;
applying axial tension on said suture filament while stabilizing said suture filament and while said second field loop surrounds said tissue structure at said proximal site so as to close said second field loop and tighten said second field knot to form a proximal ligature;
cutting said suture filament proximate said proximal ligature; and
withdrawing said suture filament through said surgical site.

22. The method of claim 21, further comprising:
transecting said tissue structure proximate said first ligature; and
withdrawing said distal end through said surgical site before said step of tying a second slidable field knot.

23. The method of claim 21, further comprising:
transecting said tissue structure proximate said second ligature; and
withdrawing said proximal end through said surgical site.

24. The method of claim 21, wherein said step of advancing said first field loop toward said distal site further comprises using an elongate tool to push said first field loop.

25. The method of claim 24, wherein said step of stabilizing said suture filament while said first field loop surrounds said tissue structure at said distal site further comprises stabilizing said elongate tool.

26. The method of claim 24, wherein said elongate tool comprises a cannula having first and second ends and a channel therethrough, said method further comprising:
threading said one end of suture filament through said channel before said step of passing said one end into said surgical port and about said tissue structure.

27. The method of claim 26, wherein said step of using an elongate tool further comprises using said first end of said cannula.

28. The method of claim 26, wherein said step of stabilizing said suture filament while said first field loop surrounds said tissue structure at said distal site further comprises stabilizing said cannula.

29. The method of claim 26, further comprising the steps of:
providing a plurality of cannulae of varying shapes and sizes; and
selecting said cannula from said plurality of cannulae.

30. The method of claim 21, wherein said step of advancing said prepared loop toward said proximal site further comprises using an elongate tool to push said prepared loop.

31. The method of claim 30, wherein said step of stabilizing said suture filament while said second field loop surrounds said tissue structure at said proximal site further comprises stabilizing said elongate tool.

32. The method of claim 30, wherein said elongate tool comprises a cannula having first and second ends and a channel therethrough, said method further comprising:
threading said one end of suture filament through said channel before said step of advancing said second field loop.

33. The method of claim 32, wherein said step of using an elongate tool further comprises using said second end of said cannula.

34. The method of claim 32, wherein said step of stabilizing said suture filament while said second field loop surrounds said tissue structure at said proximal site further comprises stabilizing said cannula.

35. The method of claim 32, further comprising the steps of:
providing a plurality of cannulae of varying shapes and sizes; and
selecting said cannula from said plurality of cannulae.

36. The method of claim 21, further comprising:
providing instructions for tying said first and second field knots.

37. The method of claim 21, wherein said steps of tying said first and second field knots further comprise at least three throws around said suture filament.

38. The method of claim 21, further comprising the steps of:
providing a plurality of suture filaments of varying types and sizes; and
selecting said suture filament from said plurality of suture filaments.

39. An apparatus for ligating an elongate tissue structure at one or more sites through a surgical opening spaced apart from at least one of said one or more sites, said apparatus comprising:
an elongate tool having a first end and a second end, said tool defining a channel therethrough, said tool being narrowed toward said ends, said tool having a length sufficient to extend from said surgical opening to each of said one or more sites; and
a suture filament having a head end and a tail end, said suture filament comprising a prepared loop in said head end secured by a slidable prepared knot tied about said suture filament near said second end of said tool, said suture filament being threaded through said channel with said tail end extending from said first end, said tail end having sufficient length to allow formation of field loop secured by a slidable field knot in said tail end.

40. The apparatus of claim 39, wherein said elongate tool has a central longitudinal axis and wherein said channel lies along said axis.

41. The apparatus of claim 39, wherein said elongate tool further comprises a first conical taper at said first end and a second conical taper at said second end, said conical tapers decreasing in diameter toward said ends.

42. The apparatus of claim 39, wherein said elongate tool comprises a cannula.

43. The apparatus of claim 39, wherein said one or more sites comprises a proximal site and a distal site.

44. The apparatus of claim 39, wherein said elongate tool further comprises:
a temporary anchor positioned and configured to exert a grasp upon said suture filament, thereby preventing movement of said suture filament through said channel,
wherein said grasp is capable of being overcome by a force exerted upon said suture filament, said force exceeding a pre-determined threshold.

45. The apparatus of claim 44, wherein said temporary anchor comprises an adhesive spot.

46. The apparatus of claim 39, wherein said sufficient length of said tail end is at least eight inches.

47. The apparatus of claim 39, wherein said surgical opening comprises a surgical port.

48. The apparatus of claim 39, wherein said surgical opening comprises a surgical incision.

49. An apparatus for ligating an elongate tissue structure at one or more sites through a surgical opening spaced apart from at least one of said one or more sites, said apparatus comprising:
an elongate tool having a first end and a second end, said tool defining a channel therethrough, said tool being narrowed toward said ends, said tool having a length sufficient to extend from said surgical opening to each of said one or more sites; and
a suture filament threaded through said channel, said suture filament having a head end and a tail end, each of said head and tail ends having sufficient length extending beyond said first and second ends of said tool, respectively, to allow formation of at least one field loop secured by a slidable field knot.

50. The apparatus of claim 49, wherein said elongate tool has a central longitudinal axis and wherein said channel lies along said axis.

51. The apparatus of claim 49, wherein said elongate tool further comprises a first conical taper at said first end and a second conical taper at said second end, said conical tapers decreasing in diameter toward said ends.

52. The apparatus of claim 49, wherein said elongate tool comprises a cannula.

53. The apparatus of claim 49, wherein said one or more sites comprises a proximal site and a distal site.

54. The apparatus of claim 49, wherein said elongate tool further comprises:
a temporary anchor positioned and configured to exert a grasp upon said suture filament, thereby preventing movement of said suture filament through said channel,
wherein said grasp is capable of being overcome by a force exerted upon said suture filament, said force exceeding a pre-determined threshold.

55. The apparatus of claim 54, wherein said temporary anchor comprises an adhesive spot.

56. The apparatus of claim 49, wherein said sufficient length extending beyond said first and second ends of said tool is at least eight inches.

57. The apparatus of claim 49, wherein said surgical opening comprises a surgical port.

58. The apparatus of claim 49, wherein said surgical opening comprises a surgical incision.
